(19) United States
(12) Patent Application Publication
(10) Pub. No.: US 2004/0002521 A1
(43) Pub. Date: Jan. 1, 2004

(54) Cyclopropanecarboxylic acid amide compound and pharmaceutical use thereof

(75) Inventors: Yukio Iino, Kawasaki-Shi (JP); Takashi Yamamoto, Kawasaki-Shi (JP); Tsuyoshi Kobayashi, Kawasaki-Shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO. INC, Tokyo, JP

(21) Appl. No.: 10/425,918

(22) Filed: Apr. 30, 2003

Related U.S. Application Data
(63) Continuation of application No. PCT/JP01/09554, filed on Oct. 31, 2001.

(30) Foreign Application Priority Data
Nov. 1, 2000 (JP) 2000-334271

(51) Int. Cl. A61K 31/445; A61K 31/4015; A61K 31/40; A61K 31/277; A61K 31/16; C07D 213/75; C07D 213/84; C 07D 2 7/34; C07C 255/50; C07C 233/34

(52) U.S. Cl. 514/344; 514/349; 514/352; 514/424; 514/426; 514/522; 514/624; 546/289; 546/297; 546/304; 548/557; 548/550; 564/190; 558/414

(57) ABSTRACT

The present invention provides an NF-kappa B activation inhibitor, an inflammatory cytokine production inhibitor, a matrix metalloprotease production inhibitor, an inflammatory cell adhesion factor expression inhibitor, and an anti-inflammatory agent, an antirheumatic agent, an immuno-suppressive agent, an antiallergic agent, an antiviral agent, or a therapeutic agent for arteriosclerosis, which comprise, as the active ingredient, a cyclopropanecarboxylic acid amide compound or a pharmaceutically acceptable salt thereof effective for the treatment of inflammatory diseases.

### BACKGROUND OF THE INVENTION

The present invention relates to a medicine for treating various inflammatory diseases.

It has been known that various kinds of inflammatory diseases, rheumatism, immunoreactive diseases, metastasis of cancer and viral diseases are caused due to, for instance, abnormal production of inflammatory cytokines and matrix metalloproteases and increase in the expression of inflammatory cell adhesion factors. Various substances or compounds have conventionally been developed as pharmaceutical agents (or medicines) for treating these diseases, but there have still been desired for the development of pharmaceutical agents having further improved efficacy, a reduced side effect and high safety.

In various chronic inflammatory diseases, mediators for inflammation such as various kinds of cytokines (in particular, inflammatory ones include, for instance, IL-1, IL-2, IL-6, IL-8 and TNF), adhesion factors and histoclastic enzymes (such as matrix metalloproteases) are continuously produced by the continuous extra-cellular stimulations and as a result, it has been recognized that the pathema of the chronic inflammatory disease is thus generated.

These mediators for inflammation are produced as a result of the activation of the genes expressing these mediators due to the extra-cellular stimulations and at this stage, a transcription factor (TF) known as NF-kappa B plays the most important role. For this reason, if the activation of this factor NF-kappa B can be inhibited, one can prevent the inflammation from enlarging and becoming chronic and it will be expected that this may be a promising method for treating inflammatory diseases such as rheumatoid arthritis and various autoimmune diseases.

In fact, glucocorticoid hormone (GC), which can strongly inhibit the activation of NF-kappa B present within cells, has been used as an intensive anti-inflammatory agent and an immuno-suppressive agent, but GC suffers from various kinds of side effects due to the hormonal effect and a rebound phenomenon and accordingly, it has presently been limited in the application as a medicine.

### DISCLOSURE OF THE INVENTION

An object of the present invention is to provide a pharmaceutical agent for treating chronic inflammatory diseases, which has a high efficacy and a considerably reduced side effect.

Another object of the present invention is to provide a compound effective for the treatment of various inflammatory diseases.

A further object of the present invention is to provide a pharmaceutical composition comprising the foregoing compound.

A still further object of the present invention is to provide an NF-kappa B activation inhibitor, which comprises the foregoing compound.

A still another object of the present invention is to provide an inflammatory cytokine production inhibitor, a matrix metalloproteases production inhibitor or an inflammatory cell adhesion factor expression inhibitor, which comprises the foregoing compound.

A still further object of the present invention is to provide an anti-inflammatory agent, an antirheumatic agent, an immunosuppressive agent, an antiallergic agent, a transplantation rejection inhibitor, a therapeutic agent for psoriatic, a cancer metastasis inhibitor, an antiviral agent, a therapeutic agent for arteriosclerosis, a therapeutic agent for ischemic reperfusion disorder or a therapeutic agent for renal failure, which comprises the foregoing compound.

A still another object of the present invention is to provide the use of the foregoing compound as an anti-inflammatory agent, an antirheumatic agent, an immunosuppressive agent, an antiallergic agent, a transplantation rejection inhibitor, a therapeutic agent for psoriatic, a cancer metastasis inhibitor, an antiviral agent, a therapeutic agent for arteriosclerosis, a therapeutic agent for ischemic reperfusion disorder or a therapeutic agent for renal failure.

The inventors of the present invention have conducted various investigations for developing a compound, which is effective for the treatment of various chronic inflammatory diseases and have a strong activity of inhibiting NF-kappa B activation, have found that the compounds represented by the following general formula (I) are quite effective for that purpose and have thus completed the present invention.

According to the present invention, there is provided an NF-kappa B activation inhibitor, an inflammatory cytokine production inhibitor, a matrix metalloproteases production inhibitor or an inflammatory cell adhesion factor expression inhibitor comprising, as the active ingredient, a cyclopropanecarboxylic acid amide compound represented by the following general formula (I) or a pharmaceutically acceptable salt thereof:

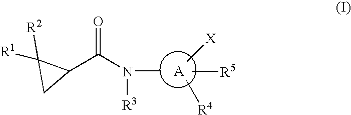

In the formula (I), R¹ and R² may be the same or different and each represents an alkyl group or a halogen atom; R³ represents a hydrogen atom or an alkyl group; A represents an aromatic ring or a heterocyclic ring; R⁴ and R⁵ may be the same or different and each represents a hydrogen atom, a halogen atom, a hydroxyl group, an alkyl group, an alkyl group having a substituent(s), an aryl group, an aryl group having a substituent(s), a mercapto group, an alkoxy group, an alkylthio group, an acyl group, an acyloxy group, an amino group, an alkylamino group, an amino protecting group-substituted amino group, a carboxyl group, an alkoxycarbonyl group, a carbamoyl group, a nitro group, a trifluoromethyl group or a cyano group; X represents a hydrogen atom, a hydroxyl group, an alkyl group having a substituent(s), an aryl group, an aryl group having a substituent(s), a mercapto group, an alkoxy group, an alkylthio group, an acyl group, an acyloxy group, an amino group, an alkylamino group, an amino protecting group-substituted amino group, a carboxyl group, an alkoxycarbonyl group, a carbamoyl group, a nitro group, a trifluoromethyl group, a cyano group or a group represented by the following general formula (II):

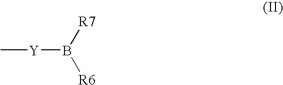

wherein —Y— represents an atomic bond, —O—, —CR⁸R⁹—, —CO—, —NR¹⁰—, —S—, —SO—, —SO₂—, —O—CO—, —CO—O—, —CO—NR¹¹—, —NR¹²—CO—, —SO₂—NR¹³—, —NR¹⁴—SO₂—, —CR¹⁵═CR¹⁶—, —CR¹⁷R¹⁸—CR¹⁹R²⁰— (wherein R⁸, R⁹, R¹⁵, R¹⁶, R¹⁷, R¹⁸, R¹⁹ and R²⁰ each represents a hydrogen atom, a halogen atom, a hydroxyl group, an alkyl group, an alkyl group having a substituent(s), a mercapto group, an alkoxy group, an alkylthio group, an acyl group, an acyloxy group, an amino group, an alkylamino group, an amino protecting group-substituted amino group, a carboxyl group, an alkoxycarbonyl group, a carbamoyl group or a cyano group; and R¹⁰, R¹¹, R¹², R¹³ and R¹⁴ each represents a hydrogen atom or an alkyl group); B represents an aromatic ring or a heterocyclic ring; R⁶ and R⁷ may be the same or different and each represents a hydrogen atom, a halogen atom, an alkyl group, an alkyl group having a substituent(s), an aryl group, an aryl group having a substituent(s), a hydroxyl group, a mercapto group, an alkoxy group, an alkylthio group, an acyl group, an acyloxy group, a carboxyl group, an alkoxycarbonyl group, a carbamoyl group, a nitro group, a trifluoromethyl group or a cyano group, provided that either R⁶ or R⁷ may be linked with A to form a ring].

Moreover, the present invention provides a cyclopropanecarboxylic acid amide or a pharmaceutically acceptable salt thereof represented by the foregoing formula (I) wherein A represents a benzene ring, a naphthalene ring, an indene ring, a pyridine ring, a dihydropyran ring, a pyridazine ring, a pyrimidine ring, a pyrazine ring, a pyrrole ring, a furan ring, a thiophene ring, an oxazole ring, an isoxazole ring, an imidazole ring, a thiazole ring, an iso-thiazole ring, a furazane ring, a pyrrolidine ring, a piperidine ring, a piperazine ring, an indole ring, an iso- indole ring, an iso-benzofuran ring, a benzothiophene ring, a benzopyrazole ring, a benzimidazole ring, a benzoxazole ring, a benzothiazole ring, a purine ring, a pyrazolopyridine ring, a quinoline ring, an iso-quinoline ring, a naphthyridine ring, a quinazoline ring, a benzodiazepine ring, a carbazole ring or a dibenzofuran ring; and B represents a benzene ring, a naphthalene ring, an indene ring, a pyridine ring, a dihydropyran ring, a pyridazine ring, a pyrimidine ring, a pyrazine ring, a pyrrole ring, a furan ring, a thiophene ring, an oxazole ring, an isoxazole ring, an imidazole ring, a thiazole ring, an iso-thiazole ring, a thiadiazole ring, a furazane ring, a pyrrolidine ring, a piperidine ring, a piperazine ring, an indole ring, an iso-indole ring, a benzofuran ring, an iso-benzofuran ring, a benzothiophene ring, a benzopyrazole ring, a benzimidazole ring, a benzoxazole ring, a benzothiazole ring, a purine ring, a pyrazolopyridine ring, a quinoline ring, an iso-quinoline ring, a naphthyridine ring, a quinazoline ring, a benzodiazepine ring, a carbazole ring or a dibenzofuran ring.

In this respect, however, when A represents a benzene ring and X represents a hydrogen atom, at least one of R⁴ and R⁵ is not a hydrogen atom; when A represents a benzene ring, X represents a hydrogen atom and one of R⁴ and R⁵ is a chlorine atom, the other of R⁴ and R⁵ is not a chlorine atom or a methyl group; and/or the following compounds (III), (IV) and (V) are excluded:

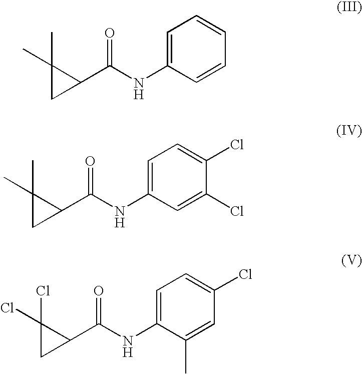

The compound represented by the foregoing general formula (I) or a salt thereof can be used as an anti-inflammatory agent, an antirheumatic agent, an immuno-suppressive agent, an antiallergic agent, a transplantation rejection inhibitor, a therapeutic agent for psoriatic, a cancer metastasis inhibitor, an antiviral agent, a therapeutic agent for arteriosclerosis, a therapeutic agent for ischemic reperfusion disorder or a therapeutic agent for renal failure.

### BEST MODE FOR CARRYING OUT THE INVENTION

The present invention will hereunder be described in more detail.

Examples of halogen atom used herein include a fluorine atom, a chlorine atom, a bromine atom and an iodine atom.

The term “alkyl group” used herein means a linear or branched alkyl group having 1 to 6 carbon atoms and specific examples thereof are methyl, ethyl, n-propyl, isopropyl, n-butyl, isobutyl, sec-butyl, tert-butyl, n-pentyl, iso-pentyl, tert-pentyl, neo-pentyl, 2-pentyl, 3-pentyl, n-hexyl and 2-hexyl groups, with methyl and ethyl groups being preferred.

Specific examples of aryl groups used herein are phenyl, indenyl, naphthyl and fluorenyl groups, with a phenyl group being preferred.

The term “alkoxy group” used herein means an alkoxy group with a linear, branched or cyclic alkyl group having 1 to 6 carbon atoms and specific examples thereof are methoxy, ethoxy, n-propoxy, isopropoxy, n-butoxy, iso-butoxy, sec-butoxy, tert-butoxy, cyclopropyloxy, cyclobutoxy, cyclopentyloxy, cyclohexyloxy and trifluoro-methoxy groups, with methoxy and ethoxy groups being preferably used.

The term “alkylthio group” used herein means an alkylthio group with a linear, branched or cyclic alkyl group having 1 to 6 carbon atoms and specific examples thereof are methylthio, ethylthio, n-propylthio, iso-propylthio, n-butylthio, iso-butylthio, sec-butylthio, tert-butylthio, cyclopropylthio, cyclobutylthio, cyclopentylthio and cyclobutylthio groups, with methylthio and ethylthio groups being preferably used.

The term “acyl group” used herein means a formyl group, an acyl group with a linear, branched or cyclic alkyl group having 1 to 6 carbon atoms or an acyl group, which may have a substituent(s). Specific examples thereof are formyl, acetyl, propionyl, butyryl, iso-butyryl, valeryl, iso-valeryl, pivaloyl, hexanoyl, acryloyl, methacryloyl, crotonoyl, iso-crotonoyl, benzoyl and naphthoyl groups, with acetyl and benzoyl groups being preferably used.

The term “acyloxy group” used herein means a formyloxy group, an acyloxy group with a linear, branched or cyclic alkyl group having 1 to 6 carbon atoms or an acyloxy group with an aryl group, which may have a substituent(s). Specific examples thereof are formyloxy, acetyloxy, propionyloxy, butyryloxy, iso-butyryloxy, valeryloxy, iso-valeryloxy, pivaloyloxy, hexanoyloxy, acryloyloxy, methacryloyloxy, crotonoyloxy, iso-crotonoyloxy, benzoyloxy and naphthoyloxy groups, with acetyloxy and benzoyloxy groups being preferably used.

The term “alkylamino group” used herein means amino groups mono-substituted or di-substituted with an alkyl group(s) and examples of such alkyl groups are those listed above in connection with the term “alkyl group”. Specific examples thereof include amino, methylamino, ethylamino, propylamine, iso-propylamino, di-methylamino, diethylamino, di-propylamino, di-isopropylamino and methyl-ethylamino groups, with amino, methylamino and dimethylamino groups being preferably used.

The term “amino protecting group” in the “amino protecting group-substituted amino group” used herein means a commonly used protecting group and it is not restricted to any particular one inasmuch as it can protect a specific amino group from any reaction. Specific examples thereof are acyl groups such as formyl, acetyl, and pivaloyl groups and alkoxycarbonyl groups such as methoxycarbonyl, ethoxycarbonyl, tert-butoxycarbonyl and fluoren-9-yl methoxycarbonyl groups. Examples thereof preferably used include acetyl, methoxycarbonyl and ethoxycarbonyl groups.

The term “alkoxycarbonyl group” used herein means an alkoxycarbonyl group with a linear, branched or cyclic alkyl group having 1 to 6 carbon atoms. Specific examples thereof are methoxycarbonyl, ethoxycarbonyl, propoxycarbonyl, iso-propoxy-carbonyl, n-butoxycarbonyl, iso-butoxycarbonyl, sec-butoxycarbonyl and tert-butoxy- carbonyl groups. Examples thereof preferably used include methoxycarbonyl and ethoxycarbonyl groups.

The term “carbamoyl group” used herein means a carbamoyl group, which may have a linear, branched or cyclic alkyl group having 1 to 6 carbon atoms on the nitrogen atom thereof. Specific examples thereof are carbamoyl, N-ethyl-carbamoyl, N,N- dimethyl-carbamoyl, N-pyrrolidyl-carbamoyl, N-piperidyl-carbamoyl and N-morpholinyl-carbamoyl groups. A carbamoyl group is preferably used in the invention.

The term “have a substituent(s)” appearing in the term “an alkyl group having a substituent(s)” used herein means that the corresponding alkyl group is substituted with at least one substituent. If the alkyl group has at least two substituents, they may be the same or different. In addition, the substituent(s) may be present on any position on the alkyl group and the positions thereof are not restricted to specific ones at all. Specific examples of such substituents are halogen atoms, hydroxyl group, alkyl groups, aryl groups, mercapto groups, alkoxy groups, alkylthio groups, acyl groups, acyloxy groups, amino groups, alkylamino groups, amino protecting group-substituted amino groups, carboxyl groups, alkoxycarbonyl groups, carbamoyl groups, nitro groups, trifluoromethyl group or cyano groups, with aryl, amino and hydroxyl groups being preferably used.

The term “have a substituent(s)” appearing in the term “an aryl group having a substituent(s)” used herein means that the corresponding aryl group is substituted with at least one substituent. If the aryl group has at least two substituents, they may be the same or different. In addition, the substituent(s) may be present on any position on the aryl group and the positions thereof are not restricted to specific ones at all. Specific examples of such substituents are halogen atoms, hydroxyl group, alkyl groups, aryl groups, mercapto groups, alkoxy groups, alkylthio groups, acyl groups, acyloxy groups, amino groups, alkylamino groups, amino protecting group-substituted amino groups, carboxyl groups, alkoxycarbonyl groups, carbamoyl groups, nitro groups, trifluoromethyl group or cyano groups, with halogen atoms, hydroxyl groups, alkoxy groups and nitro groups being preferably used.

The term “aromatic ring” used herein means a monocyclic or bicyclic aromatic ring constituted by carbon atoms and specific examples thereof are benzene rings, naphthalene rings and indene rings, with benzene rings being preferred.

The term “heterocyclic ring” used herein means a heterocyclic ring comprising one to three 5- to 7-membered rings, each constituted by, for instance, carbon atom(s) as well as nitrogen(s), oxygen(s), sulfur(s) and the like. Specific examples thereof are pyridine, dihydropyran, pyridazine, pyrimidine, pyrazine, pyrrole, furan, thiophene, oxazole, isoxazole, pyrazole, imidazole, thiazole, iso-thiazole, thiadiazole, furazane, pyrrolidine, piperidine, piperazine, indole, iso-indole, benzofuran, iso-benzofuran, benzothiophene, benzopyrazole, benzimidazole, benzoxazole, benzothiazole, purine, pyrazolopyridine, quinoline, iso-quinoline, naphthyridine, quinazoline, benzodiazepine, carbazole or dibenzofuran rings, with pyridine and piperazine rings being preferably used.

In Formula (I), X, R⁴ and R⁵ are substituents on the ring A, they may be present on any position on the ring A or the positions thereof are not restricted to specific ones.

In Formula (II), R⁶, R⁷ and R⁸ are substituents on the ring B, they may be present on any position on the ring B or the positions thereof are not restricted to specific ones.

The substituent R¹ preferably represents an alkyl group having 1 to 3 carbon atoms or a halogen atom, more preferably a methyl group or a chlorine atom and particularly preferably a methyl group.

The substituent R² preferably represents an alkyl group having 1 to 3 carbon atoms or a halogen atom, more preferably a methyl group or a chlorine atom and particularly preferably a methyl group.

The substituents R¹ and R² may be the same or different and preferably they have the same meaning.

The substituent R³ preferably represents a hydrogen atom or an alkyl group having 1 to 3 carbon atoms, more preferably a hydrogen atom or a methyl group and particularly preferably a hydrogen atom.

The aromatic or heterocyclic ring A preferably represents benzene, naphthalene, indene, pyridine, dihydropyran, pyridazine, pyrimidinepyryrazine, pyrrole, furan, thiophene, imidazole, pyrrolidine, piperidine, piperazine, indole, iso-indole, iso-benzofuran, benzopyrazole, benzimidazole, benzoxazole, benzothiazole, purine, pyrazolopyridine, quinoline, iso-quinoline, naphthyridine, quinazoline, benzodiazepine, carbazole or dibenzofuran rings, with benzene, pyridine and piperazine rings being preferred and benzene and pyridine rings being more preferred.

The substituent R⁴ preferably represents a hydrogen atom, a halogen atom, an alkyl group having 1 to 3 carbon atoms, an alkoxycarbonyl group with an alkyl group having 1 to 3 carbon atoms or a nitro group, with a hydrogen atom, a chlorine atom, a methyl group, a methoxycarbonyl group or a nitro group being more preferred and a hydrogen atom being particularly preferred.

The substituent R⁵ preferably represents a hydrogen atom, a halogen atom, an alkyl group having 1 to 3 carbon atoms, an alkoxycarbonyl group with an alkyl group having 1 to 3 carbon atoms or a nitro group, with a hydrogen atom, a chlorine atom, a methyl group, a methoxycarbonyl group or a nitro group being more preferred and a hydrogen atom being particularly preferred.

The substituents R⁴ and R⁵ may be the same or different and preferably they have the same meaning.

The substituent X preferably represents an alkoxycarbonyl group with an alkyl group having 1 to 3 carbon atoms, with a methoxycarbonyl group being more preferred.

Also preferred as the substituent X include those represented by the general formula (II).

In this respect, the divalent group —Y— preferably represents —O—, —CR⁸R⁹— (wherein both of R⁸ and R⁹ preferably represent hydrogen atoms), —CO—, —NR¹⁰— (R¹⁰ preferably represents a hydrogen atom or an alkyl group having 1 to 3 carbon atoms), —S— and —SO₂—, with —O—, —CH₂—, —CO—, —NH—, —S— and —CHCN— being more preferred.

The substituent R⁶ preferably represents a hydrogen atom, a halogen atom, an alkyl group having 1 to 3 carbon atoms, an alkoxy group having 1 to 3 carbon atoms or a nitro group, with hydrogen or chlorine atom or a methyl, methoxy or nitro group being more preferred and a hydrogen atom being particularly preferred.

The substituent R⁷ preferably represents a hydrogen atom, a halogen atom, an alkyl group having 1 to 3 carbon atoms, an alkoxy group having 1 to 3 carbon atoms or a nitro group, with hydrogen or chlorine atom or a methyl, methoxy or nitro group being more preferred and a hydrogen atom being particularly preferred.

The substituents R⁶ and R⁷ may be the same or different and preferably they have the same meaning.

The aromatic or heterocyclic ring B is preferably a benzene or pyridine ring, with a benzene ring being particularly preferred.

In Formula (I), the absolute configuration of the carbon atom in the proximity to the carbonyl group on the cyclopropyl group may be either R or S, but when both of R¹ and R² are alkyl groups, the absolute configuration thereof is more preferably S and when either of R¹ or R² represents a halogen atom, the configuration thereof is more preferably R.

A compound or a pharmaceutically acceptable salt thereof particularly preferably used in the present invention is one selected from the group consisting of those listed below:

(1) 2,2-Dimethyl-cyclopropanecarboxylic acid (4-benzylphenyl)-amide;

(2) 2,2-Dimethyl-cyclopropanecarboxylic acid p-tolylamide;

(3) 2,2-Dimethyl-cyclopropanecarboxylic acid (4-methoxycrbonyl-phenyl)-amide;

(4) 2,2-Dimethyl-cyclopropanecarboxylic acid (9-ethyl-9H-carbazol-3-yl)-amide;

(5) 2,2-Dimethyl-cyclopropanecarboxylic acid (1-benzyl-piperidin-4-yl)-amide;

(6) 2,2-Dimethyl-cyclopropanecarboxylic acid (4-phenoxy-phenyl)-amide;

(7) 2,2-Dimethyl-cyclopropanecarboxylic acid (2-phenoxy-phenyl)-amide;

(8) 2,2-Dimethyl-cyclopropanecarboxylic acid (3-phenoxy-phenyl)-amide;

(9) 2,2-Dimethyl-cyclopropanecarboxylic acid [4-(4-chlorophenoxy)-phenyl]-amide;

(10) 2,2-Dimethyl-cyclopropanecarboxylic acid [4-(2,4-dinitro-phenylamino)-phenyl]-amide;

(11) 2,2-Dimethyl-cyclopropanecarboxylic acid [4-(4-nitrophenyl-sulfonyl)-phenyl]-amide;

(12) 2,2-Dimethyl-cyclopropanecarboxylic acid (4-phenylamino-phenyl)-amide;

(13) 2,2-Dimethyl-cyclopropanecarboxylic acid (4-p-tolyloxy-phenyl)-amide;

(14) 2,2-Dimethyl-cyclopropanecarboxylic acid [4-(4-methoxy-phenylamino)-phenyl]-amide;

(15) 2,2-Dimethyl-cyclopropanecarboxylic acid (2-nitro-4-phenylcarbonyl-phenyl)-amide;

(16) 2,2-Dimethyl-cyclopropanecarboxylic acid (4-cyanophenylmethyl-phenyl)-amide;

(17) 2,2-Dimethyl-cyclopropanecarboxylic acid (3-chloro-4-cyanophenylmethyl-phenyl)-amide; and

(18) 2,2-Dimethyl-cyclopropanecarboxylic acid [4-(4-nitrophenylthio)-phenyl]-amide.

Specific examples of the “pharmaceutically acceptable salts” for the sufficiently acidic compounds of the present invention include ammonium salts, alkali metal salts (examples thereof preferably include sodium and potassium salts), alkaline earth metal salts (preferred examples thereof include calcium and magnesium salts) and salts with organic bases such as di-cyclohexyl-amine salts, benzathine salts, N-methyl-D-glucan salts, hydramine salts and salts with amino acids such as alginine and lysine. Moreover, specific examples of “pharmaceutically acceptable salts” for the sufficiently basic compounds of the present invention are acid-addition salts, for instance, salts with inorganic acids such as hydrochlorides, sulfates, nitrates and malic acid salts; and salts with organic acids such as acetic acid, lactic acid, citric acid, tartaric acid, maleic acid, fumaric acid and monomethyl sulfuric acid. These salts may optionally be water containing salts or hydrates.

Moreover, the present invention includes any isomers such as optical isomers and geometrical isomers, hydrates, solvates and those in the crystalline forms.

In this respect, as di-substituted cyclopropane compounds whose skeletons are similar to those of the compounds according to the present invention, there have been disclosed in, for instance, a laid-open patent (WO 00/15603) compounds having dimethylcyclopropane amide group on one side of a diphenylmethane skeleton and an alkylamine, amide or imide group on the other side thereof such as those represented by the following formulas (A) and (B) and this patent states that these compounds show NFkB-inhibitory effects, but these compounds differ from those disclosed in the present invention:

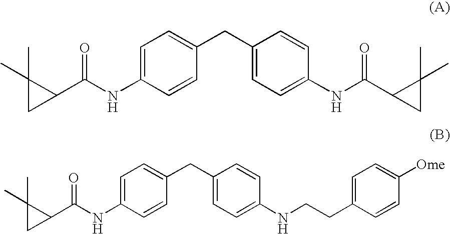

In addition, a laid-open patent (WO 99/61013) likewise discloses the foregoing compound (A), but this compound differs from that of the present invention.

Further, an article (such as Journal of Agricultural and Food Chemistry, 1967, 15: 501) discloses that the following compounds (III), (IV) and (V) have herbicidal effects, but the article never suggests the NFkB-inhibitory effects or the like disclosed in the present invention and these compounds differ from those disclosed in the present invention.

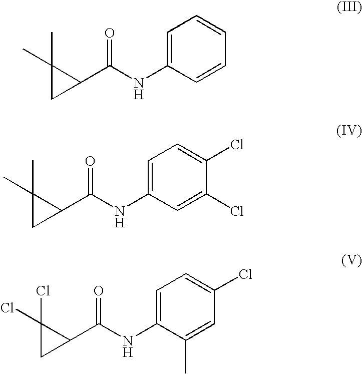

In addition, a laid-open patent (for instance, U.S. Pat. No. 5,622,989) discloses that the following compounds (C) possess an anti-inflammatory effect, but these compounds are different from those disclosed in the present invention:

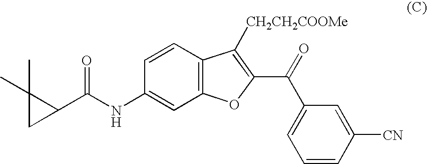

The compounds of the present invention can be synthesized according to the following methods.

For instance, the compound of Formula (I) in which A is a substituted benzene can be prepared by reacting each corresponding aniline with the corresponding acid halide such as acid chloride in the presence of a base or with a carboxylic acid in the presence of a condensing agent to obtain the intended compound as shown below:

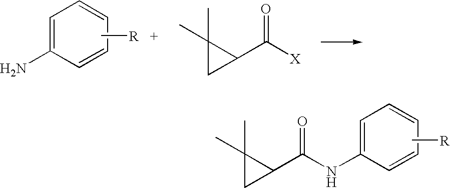

wherein R represents a substituent present on the benzene ring and X represents a hydroxyl group or a halogen atom.

In addition, the compounds disclosed in Examples according to the present invention can be prepared by applying the foregoing reaction.

In this connection, the compounds of the present invention, which can be obtained by the foregoing method, may be purified according to the methods conventionaly used in the organic synthesis such as extraction, distillation, crystallization and/or column chromatography techniques.

The resulting compounds of the present invention possess NF-kappa B activation inhibitory activities as will be discussed later and they are thus effective for the treatment of inflammatory diseases through these transcriptional factors. More specifically, the compound of the present invention is effective as an anti-inflammatory agent, an antirheumatic agent, an immunosuppressive agent, an antiallergic agent, a transplantation rejection inhibitor, a therapeutic agent for psoriatic, a cancer metastasis inhibitor, an antiviral agent, a therapeutic agent for arteriosclerosis, a therapeutic agent for ischemic reperfusion disorder or a therapeutic agent for renal failure, which can inhibit the transcription of any gene expressing, for instance, a plurality of inflammatory cytokines, matrix metalloproteases and inflammatory cell adhesion factors and which never shows any side effect such as hormonal effects.

When the compound of the present invention is used as an anti-inflammatory agent, it can be administered to a patient through oral route, intravenous injection, subcutaneous injection, or in the form of an eye drop. The dose thereof may vary depending on a variety of factors such as the symptom and age of a patient to which the compound is to be administered and the route of the administration selected, but it in general ranges from 1 to 3000 mg/kg/day.

The compound of the present invention can be formed into a pharmaceutical preparation by the usual method. The shapes of the pharmaceutical preparations may be, for instance, an injection, a tablet, a granule, a fine granule, a powder, a capsule, a cream and a suppository. In this case, the pharmaceutical preparations comprising the compounds of the present invention may comprise a variety of carriers for pharmaceutical preparations and examples thereof include lactose, glucose, D-mannitol, starch, crystalline cellulose, calcium carbonate, kaolin, starch, gelatin, hydroxypropyl cellulose, hydroxypropylmethyl cellulose, polyvinyl pyrrolidone, ethanol, carboxymethyl cellulose, calcium salt of carboxymethyl cellulose, magnesium stearate, talc, acetyl cellulose, sucrose, titanium oxide, benzoic acid, p-oxy-benzoic acid esters, sodium salt of dehydroacetic acid, gum Arabic, tragacanth, methyl cellulose, yolk, surfactants, sucrose, simple syrup, citric acid, distilled water, ethanol, glycerin, propylene glycol, macrogol, sodium hydrogen phosphate, sodium dihydrogen phosphate, sodium phosphate, glucose, sodium chloride, phenol, thimerosal, p-oxy-benzoic acid esters and sodium hydrogen-sulfite, which may be selected depending on the shape of each particular pharmaceutical preparation and admixed with the compounds of the present invention.

Moreover, the content of the effective component of the present invention present in the pharmaceutical preparation of the present invention may greatly vary depending on the shapes of the pharmaceutical preparation and is not restricted to any specific range, but it in general ranges from 0.01 to 100% by weight and preferably 1 to 100% by weight on the basis of the total weight of the composition.

### EXAMPLES

Then the present invention will hereunder be described in more detail with reference to the following Examples, but the present invention is not restricted to these specific Examples.

### Example 1

Process 1: Synthesis of Dimethyl-cyclopropanecarboxylic Acid Chloride

To a solution of dimethylcyclopropanecarboxylic acid (21.66 g, 190 mmol) in dichloromethane (100 ml), there was added N,N-dimethylformamide (10 mg), oxalyl chloride (25 g, 195 mmol) was slowly dropwise added to the resulting mixture and then the resulting reaction mixture was stirred at room temperature for 2 hours. The reaction mixture was carefully concentrated under reduced pressure (boiling point: 119° C.) to give dimethylcyclopropanecarboxylic acid chloride as an yellow oily product (20.13 g, yield: 80%).

Process 2: Synthesis of the Compound of Example 1

To a dichloromethane solution of 4-aminodiphenyl-methane (183 mg, 1 mmol), there were in order added triethylamine (about 3 equivalents) and dimethyl-cyclopropanecarboxylic acid chloride (about 2.2 equivalents) and the resulting mixture was stirred at room temperature overnight. Water was added to the mixture, the resulting mixture was extracted with ethyl acetate, the resulting extract was washed with a hydrochloric acid solution, water and a saturated aqueous sodium chloride solution, followed by drying the extract over anhydrous sodium sulfate and concentration thereof under reduced pressure. The resulting oily substance was purified by silica gel column chromatography to thus obtain a compound of Example 1 as crystals (197 mg, yield: 71%).

1H-NMR (300 MHz, CDCl3) δ=0.79-0.82 (1H, m), 1.18-1.22 (7H, m), 1.38-1.42 (1H, m), 4.76 (2H, s), 7.10-7.20 (4H, m), 7.23-7.25 (2H, m), 7.42-7.44 (3H, m).

MS (ESI) m/z: 280 (M+H)⁺.

Compounds of Examples 2 to 18 were synthesized by repeating the same procedures used in the foregoing processes 1 and 2 of Example 1.

### Example 2

In this Example, p-toluidine was used as a raw material to give a compound of Example 2 as crystals (yield: 79%).

1H-NMR (300 MHz, CDCl3) δ=0.75-0.84 (1H, m), 1.14-1.21 (7H, m), 1.39-1.41 (1H, m), 7.07 (2H, d, J=8.4Hz), 7.37 (2H, d, J=8.4 Hz). MS (ESI) m/z: 204 (M+H)⁺.

### Example 3

In this Example, p-aminobenzoic acid methyl ester was used as a raw material to give a compound of Example 3 as crystals (yield: 80%).

1H-NMR (300 MHz, CDC13) δ=0.88 (1H, d, J=7.8, 4.2 Hz), 1.21 (3H, s), 1.22-1.27 (4H, m), 1.42 (1H, dd, J=7.8, 5.1 Hz), 3.90 (3H, s), 7.60 (2H, d, J=8.7 Hz), 8.00 (2H, d, J=8.7 Hz). MS (ESI) m/z: 248 (M+H)⁺.

### Example 4

In this Example, 3-amino-9-ethyl carbazole was used as a raw material to give a compound of Example 4 as crystals (yield: 32%).

1H-NMR (300 MHz, CDCl3) δ=0.86 (1H, dd, J=4.3, 7.9 Hz), 1.23-1.30 (7H, m), 1.38-1.47 (4H, m), 4.35 (2H, q, J=6.3 Hz), 7.15-7.54 (6H, m), 8.06 (1H, d, J=7.5 Hz), 8.33 (1H, s). MS (ESI) m/z: 307 (M+H)⁺.

### Example 5

In this Example, 4-amino-1-benzyl piperazine was used as a raw material to give a compound of Example 5 as white crystals (yield: 99%).

1H-NMR (300 MHz, CDCl3) δ=0.68-0.72 (1H, m), 1.13-1.22 (6H, m), 1.38-1.51 (2H, m), 1.86-1.95 (2H, brs), 2.11 (2H, t, J=11.7 Hz), 2.78-2.84 (2H, m), 3.49 (2H, s), 3.80-3.82 (1H, m), 5.36-5.39 (1H, m), 7.26-7.30 (5H, m). MS (ESI) m/z: 287 (M+H)⁺.

### Example 6

In this Example, 4-phenoxy-aniline was used as a raw material to give a compound of Example 6 as crystals (yield: 39%).

1H-NMR (300 MHz, CDCl3) δ=0.85 (1H, m), 1.15-1.27 (7H, m), 1.37-1.42 (1H, m), 6.97 (2H, d, J=7.2 Hz), 7.06 (1H, t, J=6.5 Hz), 7.25-7.36 (4H, m), 7.47 (2H, d, J=7.5 Hz). MS (ESI) m/z: 282 (M+H)⁺.

### Example 7

In this Example, 2-phenoxy-aniline was used as a raw material to give a compound of Example 7 as crystals (yield: 42%).

1H-NMR (300 MHz, CDCl3) δ=0.81 (1H, dd, J=4.2, 7.8 Hz), 1.15-1.28 (7H, m), 1.39 (1H, dd, J=5.4, 7.8 Hz), 6.85 (2H, d, J=8.1 Hz), 6.95-7.20 (5H, m), 7.37 (2H, t, J=7.8 Hz), 7.85 (1H, s), 8.46 (1H, m). MS (ESI) m/z: 282 (M+H)⁺.

### Example 8

In this Example, 3-phenoxy-aniline was used as a raw material to give a compound of Example 8 as crystals (yield: 28%).

1H-NMR (300 MHz, CDCl3) δ=0.83 (1H, dd, J=5.2, 7.8 Hz), 1.17-1.25 (7H, m), 1.36 (1H, dd, J=6.3, 7.8 Hz), 6.71 (1H, d, J=7.3 Hz), 7.01 (2H, d, J=8.2 Hz), 7.1-7.38 (7H, m). MS (ESI) m/z: 282 (M+H)⁺.

### Example 9

In this Example, 4-(4-chlorophenoxy)-aniline was used as a raw material to give a compound of Example 9 as crystals (yield: 28%).

1H-NMR (300 MHz, CDCl3) δ=0.85 (1H, dd, J=3.7, 7.5 Hz), 1.20-1.25 (7H, m), 1.39 (1H, dd, J=5.4, 8.1 Hz), 6.90 (2H, d, J=9.0 Hz), 6.96 (2H, d, J=9.0 Hz), 7.23-7.28 (2H, m), 7.48 (2H, d, J=8.7 Hz). MS (ESI) m/z: 316 (M+H)⁺.

### Example 10

In this Example, N-(2,4-dinitrophenyl)-p-phenylene-diamine was used as a raw material to give a compound of Example 10 as crystals (yield: 71%).

1H-NMR (300 MHz, CDCl3) δ=0.81 (1H, dd, J=3.9, 7.8 Hz), 1.00 (1H, m), 1.16 (3H, s), 1.18 (3H, s), 1.68 (1H, dd, J=5.6, 7.9 Hz), 7.05 (1H, d, J=9.6 Hz), 7.43-7.45 (1H, m), 7.28 (2H, d, J=8.7 Hz), 7.72 (2H, d, J=8.4 Hz), 8.20 (1H, dd, J=2.7, 9.6 Hz), 8.89 (1H, d, J=3.0 Hz), 10.11 (0.51H, s), 10.24 (0.5H, s). MS (ESI) m/z: 369 (M−H)⁻.

### Example 11

In this Example, 4-(4-nitrophenyl-sulfonyl) aniline was used as a raw material to give a compound of Example 11 as crystals (yield: 69%).

1H-NMR (300 MHz, CDCl3) δ=0.90 (1H, dd, J=3.8, 6.8 Hz), 1.18 (6H, m), 1.23-1.26 (1H, m), 1.41 (1H, dd, J=6.1, 8.1 Hz), 7.71 (2H, d, J=9.0 Hz), 7.88 (2H, d, J=8.7 Hz), 8.08 (2H, d, J=9.0 Hz), 8.31 (2H, d, J=8.7 Hz). MS (ESI) m/z: 375 (M+H)⁺.

### Example 12

In this Example, N-phenyl-p-phenylene-diamine was used as a raw material to give a compound of Example 12 as yellow crystals (yield: 76%).

1H-NMR (300 MHz, CDCl3) δ=0.83 (1H, dd, J=4.6, 8.1 Hz), 1.12-1.24 (7H, m), 1.39 (1H, dd, J=5.1, 8.6 Hz), 5.61 (1H, s), 6.88 (1H, t, J=,7.7 Hz), 7.00 (2H, d, J=7.7 Hz), 7.04 (2H, d, J=8.7 Hz), 7.22 (2H, d, J=7.7 Hz), 7.72 (1H, d, J=8.7 Hz). MS (ESI) m/z: 281 (M+H)⁺.

### Example 13

In this Example, 4-(4-methyl-phenoxy) aniline was used as a raw material to give a compound of Example 13 as yellow oily substance (yield: 67%).

1H-NMR (300 MHz, CDCl3) δ=0.84 (1H, dd, J=4.5, 7.1 Hz), 1.19-1.20 (7H, m), 1.39 (1H, dd, J=5.8, 8.7 Hz), 2.32 (3H, s), 6.88 (2H, d, J=8.7 Hz), 6.94 (2H, d, J=8.7 Hz), 7.11 (2H, d, J=8.1 Hz), 7.45 (2H, dd, J=8.6 Hz). MS (ESI) m/z: 296 (M+H)⁺.

### Example 14

In this Example, 4-[(4-methoxyphenyl)-amino] aniline was used as a raw material to give a compound of Example 14 as crystals (yield: 31%).

1H-NMR (300 MHz, CDCl3) δ=0.82 (1H, dd, J=5.0, 9.4 Hz), 1.19-1.26 (7H, m), 1.37 (1H, dd, J=6.4, 10.0 Hz), 3.79 (3H, s), 5.43 (1H, s), 6.83-6.89 (4H, m), 7.01 (2H, d, J=6.9 Hz), 7.18 (1H, s), 7.35 (2H, d, J=8.4 Hz). MS (ESI) m/z: 311 (M+H)⁺.

### Example 15

In this Example, 4-amino-3-nitrobenzophenone was used as a raw material to give a compound of Example 15 as crystals (yield: 97%).

1H-NMR (300 MHz, CDCl3) δ=0.86 (1H, m), 1.17-1.25 (7H, m), 1.43 (1H, dd, J=5.5, 12.9 Hz), 7.47 (2H, m), 7.58 (2H, m), 7.75 (3H, m), 8.37 (1H, s), 8.77 (0.5H, s), 8.87 (0.5H, m).

### Example 16

In this Example, (4-aminophenyl)-phenyl-acetonitrile hydrochloride was used as a raw material to give a compound of Example 16 as yellow crystals (yield: 67%).

1H-NMR (300 MHz, CDCl3) δ=0.86 (1H, dd, J=4.5, 7.8 Hz), 1.12-1.24 (7H, m), 1.40 (1H, dd, J=4.8, 7.8 Hz), 5.10 (1H, s), 7.30-7.37 (8H, m), 7.54 (2H, d, J=8.1 Hz). MS (ESI) m/z: 30.5 (M+H)⁺.

### Example 17

In this Example, (4-amino-2-chlorophenyl)-phenyl-acetonitrile was used as a raw material to give a compound of Example 17 as reddish brown crystals (yield: 30%).

1H-NMR (300 MHz, CDCl3) δ=0.88 (1H, dd, J=4.2, 7.8 Hz), 1.12-1.24 (7H, m), 1.39 (1H, dd, J=5.1, 8.1 Hz), 5.59 (1H, s), 7.29-7.40 (8H, m), 7.84 (0.5H, s), 7.87 (0.5H, s). MS (ESI) m/z: 339 (M+H)⁺.

### Example 18

In this Example, 4-amino-4′-nitrodiphenyl sulfide was used as a raw material to give a compound of Example 18 as yellow crystals (yield: 30%).

1H-NMR (300 MHz, CDCl3) δ=0.91 (1H, dd, J=4.2, 7.8Hz), 1.03 (1H, dd, J=5.2, 7.2 Hz), 1.22-1.28 (6H, m), 1.44 (1H, dd, J=5.4, 7.5 Hz), 7.14 (2H, d, J=8.7 Hz), 7.44 (1H, s), 7.51 (2H, d, J=8.4 Hz), 7.65 (2H, d, J=8.4 Hz), 8.06 (1H, d, J=8.7 Hz). MS (ESI) m/z: 341 (M−H)⁻.

The compounds synthesized in Examples 1 to 18 are summarized in the following Tables 1 and 2:

TABLE 1

| Ex. | |
|---|---|
| No. | Structural formula |
| 1 | 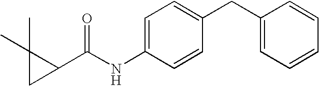 |
| 2 | 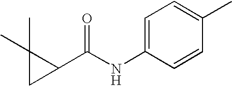 |
| 3 | 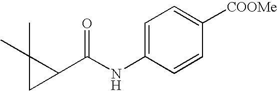 |
| 4 | 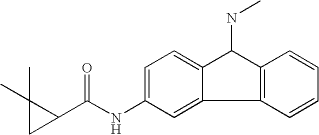 |
| 5 | 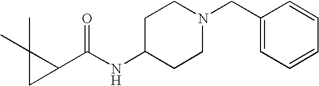 |
| 6 | 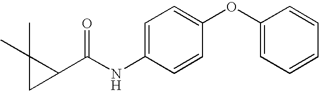 |
| 7 | 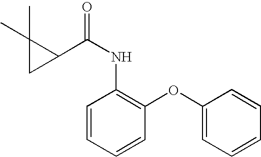 |
| 8 | 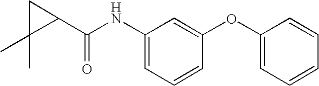 |
| 9 | 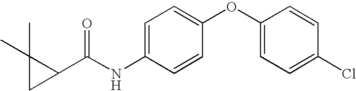 |
| 10 | 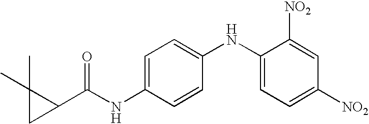 |

TABLE 2

| Ex. | |
|---|---|
| No. | Structural formula |
| 11 | 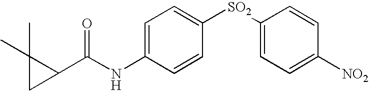 |
| 12 | 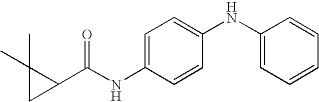 |
| 13 | 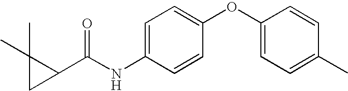 |
| 14 | 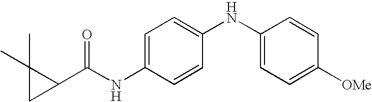 |
| 15 | 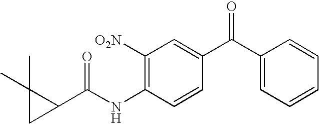 |
| 16 | 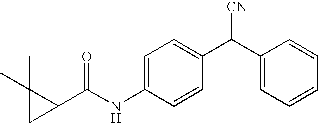 |
| 17 | 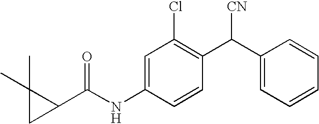 |
| 18 | 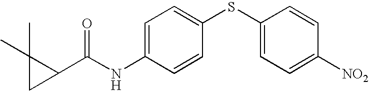 |

### Example 19

Evaluation of NF-kappa B Inhibition

In this evaluation, there were used cells obtained by stably introducing β-galactosidase (β-gal) gene of Escherichia coli, which was driven by an SV40 minimum promoter produced by fusing 6 NF-kappa B binding sequences derived from the kappa light chain enhancer of immunoglobulin arranged in a tandem to human normal umbilical venous endothelial cells (HUVEC) immortalized with an SV40 large-sized T-antigen. These cells were subjected to the subculture in an RPMI culture medium supplemented with 10% FBS (fetal bovine serum) and the cells were inoculated in wells of a 96-well plate in a concentration of 1×10⁴ cells/well on the day before the initiation of the experiments. Each candidate compound of the present invention was dissolved in DMSO in an adequate concentration and then added to each well of the 96-well plate in such a manner that the final concentration of DMSO was adjusted to a level of not more than 1%. After 30 minutes from the addition of the compound, a 1 ng/ml IL-1β was added to each well to a final concentration of 50 ng/ml to thus induce the NF-kappa B activity and the β-gal activity was determined after 16 hours therefrom. The β-gal activity was determined using a chemiluminescent substrate (Galacton-Light-Plus available from Boehringer Manheim Company), which was used according to the protocols annexed to the reagent, and Luminescencer (available from ATO Company). In this evaluation system, the β-gal activity induced by IL-1β was almost completely suppressed by glucocorticoid as an existing inhibitor for the NF-kappa B.

In the foregoing evaluation tests, the compounds of the present invention showed desired inhibitory effects.

The following Table 3 shows the results obtained in these tests for the evaluation of the compounds of the present invention.

TABLE 3

| Candidate Compound | NF-kappa B Inhibitory Activity: IC50 |
|---|---|
| Tested | (μg/ml) |
| Compound of Example 1 | 3 |
| Compound of Example 2 | 3 |
| Compound of Example 4 | 2 |
| Compound of Example 9 | 5 |
| Compound of Example 12 | 2 |

In the foregoing evaluation tests, the compounds of the present invention showed desired inhibitory effects.

As will be clear from the results described above, the compounds of the present invention possess the NF-kappa B activation inhibitory activity and they are thus effective for the treatment of inflammatory diseases through these transcriptional factors. More specifically, the compound of the present invention is effective as an anti-inflammatory agent, an antirheumatic agent, an immunosuppressive agent, an antiallergic agent, a transplantation rejection inhibitor, a therapeutic agent for psoriatic, a cancer metastasis inhibitor, an antiviral agent, a therapeutic agent for arteriosclerosis, a therapeutic agent for ischemic reperfusion disorder or a therapeutic agent for renal failure, which can inhibit the transcription of any gene expressing, for instance, a plurality of inflammatory cytokines, matrix metalloproteases and inflammatory cell adhesion factors and which never shows any side effect such as hormonal effects.

What is claimed is:

1. An NF-kappa B activation inhibitor, an inflammatory cytokine production inhibitor, a matrix metalloprotease production inhibitor or an inflammatory cell adhesion factor expression inhibitor comprising, as the active ingredient, a cyclopropanecarboxylic acid amide compound represented by the following general formula (I) or a pharmaceutically acceptable salt thereof: 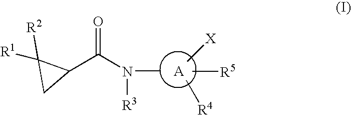
wherein R¹ and R² may be the same or different and each represents an alkyl group or a halogen atom; R³ represents a hydrogen atom or an alkyl group; A represents an aromatic ring or a heterocyclic ring;
R⁴ and R⁵ may be the same or different and each represents a hydrogen atom, a halogen atom, a hydroxyl group, an alkyl group, an alkyl group having a substituent(s), an aryl group, an aryl group having a substituent(s), a mercapto group, an alkoxy group, an alkylthio group, an acyl group, an acyloxy group, an amino group, an alkylamino group, an amino protecting group-substituted amino group, a carboxyl group, an alkoxycarbonyl group, a carbamoyl group, a nitro group, a trifluoromethyl group or a cyano group; X represents a hydrogen atom, a hydroxyl group, an alkyl group having a substituent(s), an aryl group, an aryl group having a substituent(s), a mercapto group, an alkoxy group, an alkylthio group, an acyl group, an acyloxy group, an amino group, an alkylamino group, an amino protecting group-substituted amino group, a carboxyl group, an alkoxycarbonyl group, a carbamoyl group, a nitro group, a trifluoromethyl group, a cyano group or a group represented by the following general formula (II): 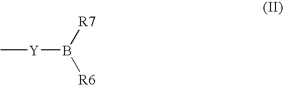
wherein —Y— represents an atomic bond, —O—, —CR⁸R⁹—, —CO—, —NR¹⁰—, —S—, —SO—, —SO₂—, —O—CO—, —CO—O—, —CO—NR¹¹—, —NR¹²—CO—, —SO₂—, —NR¹³—, —NR¹⁴—SO₂—, —CR¹⁵═CR¹⁶—, —CR¹⁷R¹⁸—CR¹⁹R²⁰— (wherein R⁸, R⁹, R¹⁵, R¹⁶, R¹⁷, R¹⁸, R¹⁹ and R²⁰ each represents a hydrogen atom, a halogen atom, a hydroxyl group, an alkyl group, an alkyl group having a substituent(s), a mercapto group, an alkoxy group, an alkylthio group, an acyl group, an acyloxy group, an amino group, an alkylamino group, an amino protecting group-substituted amino group, a carboxyl group, an alkoxycarbonyl group, a carbamoyl group or a cyano group; and R¹⁰, R¹¹, R¹², R¹³ and R¹⁴ each represents a hydrogen atom or an alkyl group); B represents an aromatic ring or a heterocyclic ring; R⁶ and R⁷ may be the same or different and each represents a hydrogen atom, a halogen atom, an alkyl group, an alkyl group having a substituent(s), an aryl group, an aryl group having a substituent(s), a hydroxyl group, a mercapto group, an alkoxy group, an alkylthio group, an acyl group, an acyloxy group, a carboxyl group, an alkoxycarbonyl group, a carbamoyl group, a nitro group, a trifluoromethyl group or a cyano group, provided that either R⁶ or R⁷ may be linked with A to form a ring].

2. A cyclopropanecarboxylic acid amide or a pharmaceutically acceptable salt thereof represented by the general formula (I) as set forth in claim 1, wherein A represents a benzene ring, a naphthalene ring, an indene ring, a pyridine ring, a dihydropyran ring, a pyridazine ring, a pyrimidine ring, a pyrazine ring, a pyrrole ring, a furan ring, a thiophene ring, an oxazole ring, an isoxazole ring, an imidazole ring, a thiazole ring, an iso-thiazole ring, a furazane ring, a pyrrolidine ring, a piperidine ring, a piperazine ring, an indole ring, an iso-indole ring, an iso-benzofuran ring, a benzothiophene ring, a benzopyrazole ring, a benzimidazole ring, a benzoxazole ring, a benzothiazole ring, a purine ring, a pyrazolopyridine ring, a quinoline ring, an iso-quinoline ring, a naphthyridine ring, a quinazoline ring, a benzodiazepine ring, a carbazole ring or a dibenzofuran ring; and B represents a benzene ring, a naphthalene ring, an indene ring, a pyridine ring, a dihydropyran ring, a pyridazine ring, a pyrimidine ring, a pyrazine ring, a pyrrole ring, a furan ring, a thiophene ring, an oxazole ring, an isoxazole ring, an imidazole ring, a thiazole ring, an iso-thiazole ring, a thiadiazole ring, a furazane ring, a pyrrolidine ring, a piperidine ring, a piperazine ring, an indole ring, an iso-indole ring, a benzofuran ring, an iso-benzofuran ring, a benzothiophene ring, a benzopyrazole ring, a benzimidazole ring, a benzoxazole ring, a benzothiazole ring, a purine ring, a pyrazolopyridine ring, a quinoline ring, an iso-quinoline ring, a naphthyridine ring, a quinazoline ring, a benzodiazepine ring, a carbazole ring or a dibenzofuran ring, provided that
(i) when A represents a benzene ring and X represents a hydrogen atom, at least one of R⁴ and R⁵ is not a hydrogen atom; when A represents a benzene ring, X represents a hydrogen atom and one of R⁴ and R⁵ is a chlorine atom, the other of R⁴ and R⁵ is not a chlorine atom or a methyl group; and/or
(ii) the following compounds (III), (IV) and (V) are excluded:

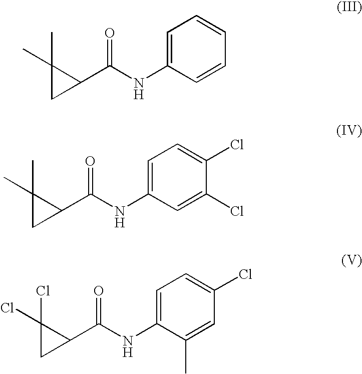

3. The cyclopropanecarboxylic acid amide or the pharmaceutically acceptable salt thereof as set forth in claim 2, wherein A represents a benzene, naphthalene, indene, pyridine, dihydropyran, pyridazine, pyrimidine, pyrazine, pyrrole, furan, thiophene, imidazole, pyrrolidine, piperidine, piperazine, indole, iso-indole, iso-benzofuran, benzopyrazole, benzimidazole, benzoxazole, benzothiazole, purine, pyrazolopyridine, quinoline, iso-quinoline, naphthyridine, quinazoline, benzodiazepine, carbazole or dibenzofuran ring.

4. The cyclopropanecarboxylic acid amide or the pharmaceutically acceptable salt thereof as set forth in claim 2, wherein when A represents a benzene ring and X represents a hydrogen atom, at least one of R⁴ and R⁵ is not a hydrogen atom; and when A represents a benzene ring, X represents a hydrogen atom and one of R⁴ and R⁵ is a chlorine atom, the other of R⁴ and R⁵ is not a chlorine atom or a methyl group.

5. The cyclopropanecarboxylic acid amide or the pharmaceutically acceptable salt thereof as set forth in claim 2, wherein it is not a compound represented by the following formula (III), (IV) or (V):

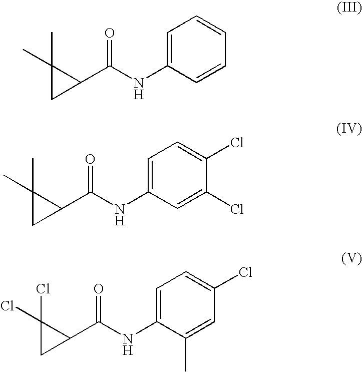

6. The cyclopropanecarboxylic acid amide or the pharmaceutically acceptable salt thereof as set forth in claim 2, wherein R³ in the general formula (I) represents a hydrogen atom or an alkyl group having 1 to 3 carbon atoms.

7. The cyclopropanecarboxylic acid amide or the pharmaceutically acceptable salt thereof as set forth in claim 6, wherein R¹ and R² in the general formula (I) each represents an alkyl group having 1 to 3 carbon atoms or a halogen atom.

8. The cyclopropanecarboxylic acid amide or the pharmaceutically acceptable salt thereof as set forth in claim 7, wherein A in the general formula (I) represents a benzene, pyridine or piperidine ring.

9. The cyclopropanecarboxylic acid amide or the pharmaceutically acceptable salt thereof as set forth in claim 8, wherein the divalent group —Y— represents —O—, —CR⁸R⁹—, —CO—, —NR¹⁰—, —S— or —SO₂—.

10. The cyclopropanecarboxylic acid amide or the pharmaceutically acceptable salt thereof as set forth in claim 2, wherein it is a member selected from the group consisting of the compounds below:
(1) 2,2-Dimethyl-cyclopropanecarboxylic acid (4-benzylphenyl)-amide;
(2) 2,2-Dimethyl-cyclopropanecarboxylic acid p-tolylamide;
(3) 2,2-Dimethyl-cyclopropanecarboxylic acid (4-methoxycrbonyl-phenyl)-amide;
(4) 2,2-Dimethyl-cyclopropanecarboxylic acid (9-ethyl-9H-carbazol-3-yl)-amide;
(5) 2,2-Dimethyl-cyclopropanecarboxylic acid (1-benzyl-piperidin-4-yl)-amide;
(6) 2,2-Dimethyl-cyclopropanecarboxylic acid (4-phenoxy-phenyl)-amide;
(7) 2,2-Dimethyl-cyclopropanecarboxylic acid (2-phenoxy-phenyl)-amide;
(8) 2,2-Dimethyl-cyclopropanecarboxylic acid (3-phenoxy-phenyl)-amide;
(9) 2,2-Dimethyl-cyclopropanecarboxylic acid [4-(4-chlorophenoxy)-phenyl]-amide;
(10) 2,2-Dimethyl-cyclopropanecarboxylic acid [4-(2,4-dinitro-phenylamino)-phenyl]-amide;
(11) 2,2-Dimethyl-cyclopropanecarboxylic acid [4-(4-nitrophenyl-sulfonyl)-phenyl]-amide;
(12) 2,2-Dimethyl-cyclopropanecarboxylic acid (4-phenylamino-phenyl)-amide;
(13) 2,2-Dimethyl-cyclopropanecarboxylic acid (4-p-tolyloxy-phenyl)-amide;
(14) 2,2-Dimethyl-cyclopropanecarboxylic acid [4-(4-methoxy-phenylamino)-phenyl]-amide;
(15) 2,2-Dimethyl-cyclopropanecarboxylic acid (2-nitro-4-phenylcarbonyl-phenyl)-amide;
(16) 2,2-Dimethyl-cyclopropanecarboxylic acid (4-cyanophenylmethyl-phenyl)-amide;
(17) 2,2-Dimethyl-cyclopropanecarboxylic acid (3-chloro-4-cyanophenylmethyl-phenyl)-amide; and
(18) 2,2-Dimethyl-cyclopropanecarboxylic acid [4-(4-nitrophenylthio)-phenyl]-amide.

11. The cyclopropanecarboxylic acid amide or the pharmaceutically acceptable salt thereof as set forth in claim 2, wherein the absolute configuration of the carbon atom in the proximity to the carbonyl group on the cyclopropyl group is S.

12. The cyclopropanecarboxylic acid amide or the pharmaceutically acceptable salt thereof as set forth in claim 2, wherein the absolute configuration of the carbon atom in the proximity to the carbonyl group on the cyclopropyl group is R.

13. A pharmaceutical composition comprising, as the active ingredient, a cyclopropanecarboxylic acid amide or a pharmaceutically acceptable salt thereof as set forth in claim 2.

14. An NF-kappa B activation inhibitor comprising, as the active ingredient, a cyclopropanecarboxylic acid amide or a pharmaceutically acceptable salt thereof as set forth in claim 2.

15. An inflammatory cytokine production inhibitor, a matrix metalloprotease production inhibitor or an inflammatory cell adhesion factor expression inhibitor comprising, as the active ingredient, a cyclopropanecarboxylic acid amide or a pharmaceutically acceptable salt thereof as set forth in claim 2.

16. An anti-inflammatory agent, an antirheumatic agent, an immuno-suppressive agent, an antiallergic agent, a transplantation rejection inhibitor, a therapeutic agent for psoriatic, a cancer metastasis inhibitor, an antiviral agent, a therapeutic agent for arteriosclerosis, a therapeutic agent for ischemic reperfusion disorder or a therapeutic agent for renal failure comprising the compound represented by the general formula (I) as set forth in claim

17. An anti-inflammatory agent, an antirheumatic agent, an immuno-suppressive agent, an antiallergic agent, a transplantation rejection inhibitor, a therapeutic agent for psoriatic, a cancer metastasis inhibitor, an antiviral agent, a therapeutic agent for arteriosclerosis, a therapeutic agent for ischemic reperfusion disorder or a therapeutic agent for renal failure comprising the compound as set forth in claim 2.

18. Use of a compound represented by the general formula (I) as set forth in claim 1 as an anti-inflammatory agent, an antirheumatic agent, an immuno-suppressive agent, an antiallergic agent, a transplantation rejection inhibitor, a therapeutic agent for psoriatic, a cancer metastasis inhibitor, an antiviral agent, a therapeutic agent for arteriosclerosis, a therapeutic agent for ischemic reperfusion disorder or a therapeutic agent for renal failure
